(19) United States
(12) Patent Application Publication
(10) Pub. No.: US 2008/0279970 A1
(43) Pub. Date: Nov. 13, 2008

(54) Pharmaceutical composition for treating parotid gland tumor, cerebroma and renal cyst

(76) Inventor: Jian Zhang, Heze (CN)

Correspondence Address: ANNA WU, P.O. BOX 93286, CITY OF INDUSTRY, CA, 91715, US

(21) Appl. No.: 11/705,220

(22) Filed: May 11, 2007

(51) Int. Cl. A61K 36/536; A61K 36/808; A61P 35/00; A61P 43/00

(52) U.S. Cl. 424/773

(57) ABSTRACT

A Chinese herb pharmaceutical composition for treating tumor is disclosed, wherein the composition is prepared by a predetermined amount of Common Selfheal, and a predetermined amount of Radixscrophulariae.

### BACKGROUND OF THE PRESENT INVENTION

1. Field of Invention

The present invention relates to manufacturing method and use of pharmaceutical composition, and more particularly to a Chinese herb composition and the manufacturing method of such for treating parodied gland tumor, cerebrum and renal cyst.

2. Description of Related Arts

For years, scientists have been seeking effective drugs for treating tumors with minimized side effects. Conventionally, surgery or γ-knife stereo tactic radiotherapy are preferred options for treating tumors. However, the subsequent radiotherapy, chemotherapy and radiotherapy would generate considerable side effects as well as unbearable suffering for patients. Needless to say, how to overcome such side effects has became a long time challenge within the art worldwide. On the other hand, traditional Chinese medicine therapy have been proven with huge advantage on much much-reduced side effects in treating tumors and cancers, and more importantly with low costs.

Recently, it is witnessed that some traditional Chinese medicine been disclosed for treating tumors and cancers, such as Chinese Patent Publication 96105287.2 detailed Chinese herb composition for treating cancers, such composition comprises Chinesis herb Prunella, Scrophulariaceae with a total of 45 medicinal herbs; what is more, Chinese Patent Publication 94110526 has disclosed a kind of drug for treating thyroid cancer, wherein such drug contains at least six Chinese herbs including Common Selfheal and Radixscrophulariae.

Nevertheless, a major drawback of traditional Chinese herb medicine is that the formulas are too complicated and too many sorts of herbs would be involved. Until now, there are no clear boundaries between treating effects of different herbs. In other words, some herbs would cause side effects or unnecessary burdens to certain organs. Therefore, too many herbs within a Chinese herb composition would cause some unnecessary burdens to the patients.

It would be highly desirable to develop a kind of Chinese herb composition for treating tumors as well as cancers, wherein such Chinese herb composition would involve much less herb combination to an acceptable extent.

### SUMMARY OF THE PRESENT INVENTION

Accordingly, a main object of the present invention is to provide a Chinese herb pharmaceutical composition for the treating tumors, wherein such composition would generate as less side effects as possible.

Another object of the present invention is to provide a low-cost, easy-to-manufacture, and easy-to-market Chinese herb pharmaceutical composition for treating tumors.

Another object of the present invention is to provide a Chinese herb pharmaceutical composition for treating parodied gland tumor, encephaloma and renal cyst, wherein such composition consists less herb combinations.

Another object of the present invention is to provide a Chinese herb pharmaceutical composition for treating parodied gland tumor, encephaloma and renal cyst, wherein Common Selfheal and Radixscrophulariae are used together as a tumor cell propagation inhibitor for curing the tumor.

Another object of the present invention is to provide a Chinese herb pharmaceutical composition for treating parodied gland tumor, encephaloma and renal cyst, wherein the inflammation could be significantly reduced.

Accordingly, in order to accomplish the above objects, the present invention provides a Chinese herb pharmaceutical composition for treating parodied gland tumor, cerebrum, and renal cyst, wherein the composition comprises a predetermined amount of common selfheal and a predetermined amount of Radixscrophulariae.

### BRIEF DESCRIPTION OF THE DRAWINGS

FIG. 1 is an illustration of herb Radixscrophulariae (known in Chinese as Xuan Shen) in natural form.

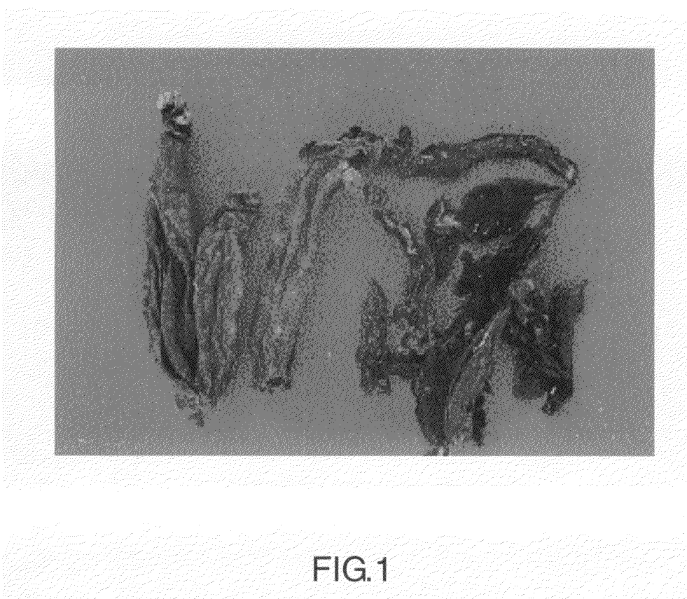

FIG. 2 is an illustration of herb common selfheal (known in Chinese as Xia Ku Cao) in natural form.

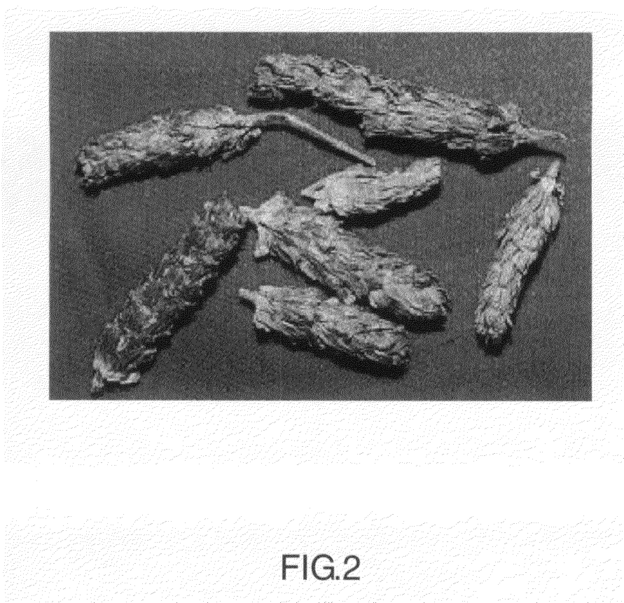

### DETAILED DESCRIPTION OF THE PREFERRED EMBODIMENT

The present invention provides a Chinese herb pharmaceutical composition for treating parodied gland tumor, encephaloma and renal cyst, wherein the composition is selected from an herb group consisting of Common Selfheal and Radixscrophulariae.

A predetermined amount of Common Selfheal and Radixscrophulariae are selected for preparing the composition. Preferably, the weight ratio of Common Selfheal is ranged from 93.75% to 95.9% within the composition and the weight ratio of Radixscrophulariae is ranged betveen 4.1% to 6.25%.

According to Chinese pharmacology, Seltheal (known as Chinese herb Xia ku cao) is of cold property and tasted bitter, functioned for lowering stomach fever, curing hypertention, headache, dizziness, wherein the Common Selfheal is provided for cold, bitter, lowering stomach fever, curing hypertension, headache and dizziness, eyes sensitivity to light, tears, painful at night, breast illness, and lowering blood pressure. Meanwhile, Radixscrophulariae (known as Chinese herb Xuan Shen) is of cold property too, and tasted bitter. It is functioned for cooling blood, promoting Yin, cleaning toxin and heat, soothing the throat, and treating eyes sensitivity to lights, tears, and so on.

According to the preferred embodiment of the present invention, the pharmaceutical composition further comprises one or more pharmaceutically acceptable excipients or one or more additional active pharmaceutical ingredients.

### EXAMPLES

The present invention will be further described by way of examples, which however should not be construed as limiting the invention.

### Example 1

Pills Preparation

The respective ingredients, Common Selfheal and Radixscrophulariae are prepared according to the predetermined weight ratios. For instance, the weights of extract ingredients drawn from respective medical herbs per g of a pill preparation in the form of powders or granules are: Common Selfheal, 38 g; and Radixscrophulariae, 2 g.

### Example 2

Capsules Preparation

The respective ingredients, Common Selfheal and Radixscrophulariae are prepared according to the predetermined weight ratios for treating parodied gland tumor, encephaloma and renal cyst: The weights of extract ingredients drawn from respective medical herbs per g of a capsules preparation in the form of powders or granules are: Common Selfheal, 35 g; and Radixscrophulariae, 1.5 g. First of all, Selfheal and Radixscrophulariae are mixed and grinded, afterwards, such mixture are filtered through a 40-mesh sieve to be applied into capsules.

### Example 3

Capsules Preparation

The respective ingredients, Common Selfheal and Radixscrophulariae are prepared according to the predetermined weight ratios for treating parodied gland tumor, encephaloma and renal cyst: The weights of extract ingredients drawn from respective medical herbs per g of a capsules preparation in the form of powders or granules are: Common Selfheal, 45 g; and Radixscrophulariae, 3 g. First of all, Selfheal and Radixscrophulariae are mixed and grinded, afterwards, such mixture are filtered through a 40-mesh sieve to be applied into capsules.

### Example 4

Decoction Preparation

The respective ingredients, Common Selfheal and Radixscrophulariae are prepared according to the predetermined weight ratios for treating parodied gland tumor, encephaloma and renal cyst: The weights of extract ingredients drawn from respective medical herbs per g of a capsules preparation in the form of powders or granules are: Common Selfheal, 40 g; and Radixscrophulariae, 2.5 g.

Clinical Observation and Analysis

203 patients of parodied gland tumor, encephaloma and renal cyst are administered with above mentioned composition. It is seen that the tumor size had been reduced 30 percent after the first treating period, 70 percent after the second treating period, and completely removal after the third treating period respectively.

It is noted that Chinese traditional medicinal preparation is a topical treatment that is to applied directly to a parodied gland tumor, encephaloma and renal cyst, or the like in order to promote healing thereof. Here, two primary herbs, Common Selfheal and Radixscrophulariae, are applied for preventing tumefaction. Furthermore, the composition of the present invention could be added with one or more secondary Chinese herbs for strengthening the function.

The composition in this invention encompasses not only medicines which exhibit a tumor cell killing effect by themselves, but also those which do not exhibit a tumor cell killing effect by themselves but suppress the propagation of tumor cells or contribute in the treatment of tumor when used in combination with other antitumor drugs, thereby allowing to fully exhibit their own tumor cell killing effects.

There is nothing special in the manner of administration when the medicine of the present invention is used in combination with other known antitumor drugs. However, the medicine of the present invention is preferably administered to a patient simultaneously with or prior to the other antitumor drugs.

It is worth to mention that the dose of the medicine for the treatment of tumor according to the present invention varies depending on the symptom, age, body weight, etc. of the patient.

It will thus be seen that the objects of the present invention have been fully and effectively accomplished. Its embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A pharmaceutical composition for treating parodied gland tumor, cerebrum and renal cyst, wherein said composition comprises common selfheal and Radixscrophulariae.

2. The composition, as recited in claim 1, comprising medicine according to claim 1, wherein a weight ratio of said Common Selfheal is ranged from 93.75% to 95.9% within said the composition and a weight ratio of said Radixscrophulariae is ranged between 4.1% to 6.25%.

3. The medicine according to claim 2, wherein said composition is prepared by the following steps:
a. Mixing said Common Selfheal and said Radixscrophulariae into a mixture according said weight ratio of said composition;
b. Grinding said mixture; and
c. Applying said mixture into oral form for administration.

4. The composition, as recited in claim 3, wherein said oral form is selected from a group consisting of Pills, Capsules and Decoction.

5. The composition, as recited in claim 1, further comprising one or more pharmaceutically acceptable excipients.

6. The composition, as recited in claim 2, further comprising one or more pharmaceutically acceptable excipients.

7. The composition, as recited in claim 3, further comprising one or more pharmaceutically acceptable excipients.

8. The composition, as recited in claim 4, further comprising one or more pharmaceutically acceptable excipients.
